(19) United States
(12) Patent Application Publication
(10) Pub. No.: US 2012/0253146 A1
(43) Pub. Date: Oct. 4, 2012

(54) Optical Instrument With Audio Band Frequency Response

(75) Inventor: Daniel Lisogurski, Boulder, CO (US)

(73) Assignee: Nellcor Puritan Bennett LLC, Boulder, CO (US)

(21) Appl. No.: 13/076,199

(22) Filed: Mar. 30, 2011

(51) Int. Cl. A61B 5/145

(52) U.S. Cl. 600/310

(57) ABSTRACT

A system and method for determining physiological parameters of a patient based on light transmitted through the patient. A light drive signal may be generated by an audio codec in a processor and utilized to generate the light transmitted through the patient. Additionally, the processor may calculate physiological parameters of the patient based on digital data signals converted in the audio codec that are indicative of absorption of light in the patient.

### BACKGROUND

The present disclosure relates generally to medical devices and, more particularly, to generation and sampling of light for photoplethysmography-based systems.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

One technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed or scattered. More specifically, the light passed through the tissue is typically selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

Pulse oximetry is a specific application in the field of photoplethysmography, which relates to analysis of the optical measurement of the change in an organ's volume (such as the change in diameter of an artery due to blood pulsation). While embodiments below may relate to pulse oximetry, it should be understood that the techniques disclosed herein are equally applicable to other photoplethysmography signals and measurements.

Manufacture of medical monitoring devices, such as those discussed above, may utilize a multitude of independent circuits, processors, and other electronic components. Use of these various electronic components may increase the overall size, complexity, and cost of the medical monitoring device. Accordingly, it may be desirable to reduce the number of electronic components utilized in a medical monitoring device to, for example, lower cost, complexity, power consumption, and/or size of the medical monitor.

Prior art devices have employed audio coder-decoders (“codecs”) in order to process pulse oximetry signals. However, these prior art devices provided signals to the codecs that were outside the codec's input frequency bandwidth (e.g., square waves). As a result, the codec's output signal was distorted relative to its input, amongst other problems. Moreover, prior art devices used codecs provided in discrete packages, resulting in increased cost, power consumption, device size, and/or device complexity.

### BRIEF DESCRIPTION OF THE DRAWINGS

Advantages of the disclosed techniques may become apparent upon reading the following detailed description and upon reference to the drawings in which:

FIG. 1 illustrates a perspective view of a pulse oximeter in accordance with an embodiment;

FIG. 2 illustrates a simplified block diagram of a pulse oximeter in FIG. 1, according to an embodiment;

FIG. 3 illustrates a block diagram of an emitter and a digital to analog converter of FIG. 2, according to an embodiment;

FIG. 4 illustrates an illustration of a waveform generated by the digital to analog converter of FIG. 3, according an embodiment; and

FIG. 5 illustrates a block diagram of an emitter and a digital to analog converter of FIG. 2, according to another embodiment.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Physiological monitors may receive data signals, calculate physiological parameters of a patient based on the data signal, and display the results of this calculation. To reduce size, cost, or power consumption or simplify the monitors, electronic components that perform a plurality of operations may be utilized. For example, a processor that includes an audio codec may perform the functions of traditional processor, light drive circuit, and analog-to-digital converter. That is, a processor that includes an audio codec may perform the functions of a plurality of typically stand alone devices. For example, the audio codec may control light drive signals that may be utilized by a photoplethysmography sensor to generate light; it may receive light signals from that same sensor and convert the signals from analog to digital signals; and the corresponding processor may utilize these converted digital signals to determine physiological parameters of a patient. By using a single processor to perform multiple functions in a physiological monitors the overall size, power consumption, complexity, and cost may be reduced.

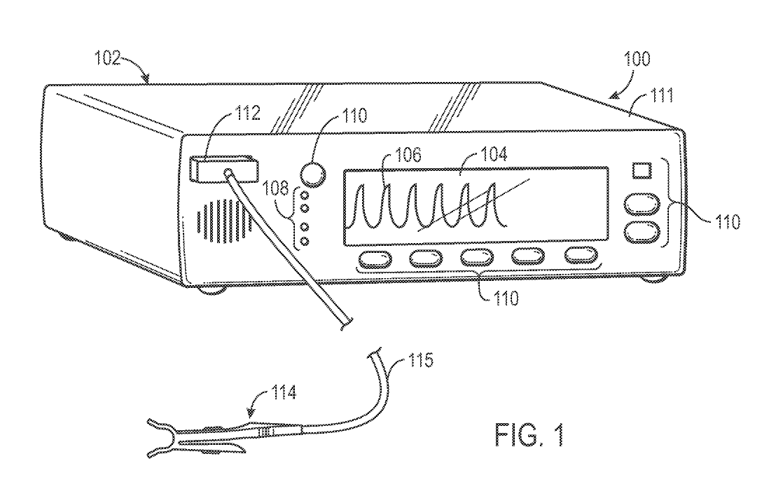

Turning to FIG. 1, a perspective view of a medical device is illustrated in accordance with an embodiment. The medical device may be a pulse oximeter 100. The pulse oximeter 100 may include a monitor 102, such as those available from Nellcor Puritan Bennett LLC. The monitor 102 may be configured to display calculated parameters on a display 104. As illustrated in FIG. 1, the display 104 may be integrated into the monitor 102. However, the monitor 102 may be configured to provide data via a port to a display (not shown) that is not integrated with the monitor 102. The display 104 may be configured to display computed physiological data including, for example, an oxygen saturation percentage, a pulse rate, and/or a plethysmographic waveform 106. As is known in the art, the oxygen saturation percentage may be a functional arterial hemoglobin oxygen saturation measurement in units of percentage SpO₂, while the pulse rate may indicate a patient's pulse rate in beats per minute. The monitor 102 may also display information related to alarms, monitor settings, and/or signal quality via indicator lights 108.

To facilitate user input, the monitor 102 may include a plurality of control inputs 110. The control inputs 110 may include fixed function keys, programmable function keys, and soft keys. Specifically, the control inputs 110 may correspond to soft key icons in the display 104. Pressing control inputs 110 associated with, or adjacent to, an icon in the display may select a corresponding option. The monitor 102 may also include a casing 111. The casing 111 may aid in the protection of the internal elements of the monitor 102 from damage.

The monitor 102 may further include a sensor port 112. The sensor port 112 may allow for connection to an external sensor 114, via a cable 115 which connects to the sensor port 112. Alternatively, the external sensor 114 may be wirelessly coupled the monitor 102. Furthermore, the sensor 114 may be of a disposable or a non-disposable type. The sensor 114 may obtain readings from a patient, which can be used by the monitor to calculate certain physiological characteristics such as the blood-oxygen saturation of hemoglobin in arterial blood, the volume of individual blood pulsations supplying the tissue, and/or the rate of blood pulsations corresponding to each heartbeat of a patient.

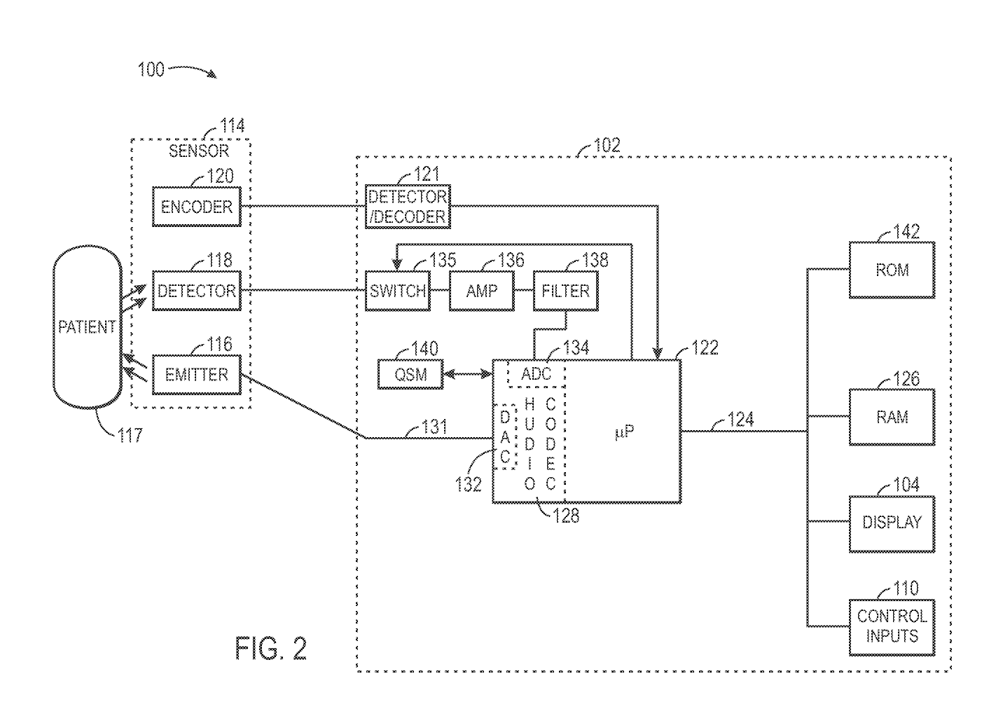

Turning to FIG. 2, a simplified block diagram of a pulse oximeter 100 is illustrated in accordance with an embodiment. Specifically, certain components of the sensor 114 and the monitor 102 are illustrated in FIG. 2. The sensor 114 may include an emitter 116, a detector 118, and an encoder 120. It should be noted that the emitter 116 may be capable of emitting at least two wavelengths of light, e.g., red and infrared (IR) light, into the tissue of a patient 117 to calculate the patient's 117 physiological characteristics, where the red wavelength may be between about 600 nanometers (nm) and about 700 nm, and the IR wavelength may be between about 800 nm and about 1000 nm. The emitter 116 may include a single emitting device, for example, with two light emitting diodes (LEDs) or the emitter 116 may include a plurality of emitting devices with, for example, multiple LEDs at various locations. For measuring certain physiological parameters, the emitter 116 may include a single LED. Regardless of the number of emitting devices, the emitter 116 may be used to measure, for example, water fractions, hematocrit, or other physiological parameters of the patient 117. It should be understood that, as used herein, the term “light” may refer to one or more of ultrasound, radio, microwave, millimeter wave, infrared, visible, ultraviolet, gamma ray, or X-ray electromagnetic radiation, and may also include any wavelength within the radio, microwave, infrared, visible, ultraviolet, or X-ray spectra, and that any suitable wavelength of light may be appropriate for use with the present disclosure.

In one embodiment, the detector 118 may include one or more detector elements that may be capable of detecting light at various intensities and wavelengths. In operation, light enters the detector 118 after passing through the tissue of the patient 117. The detector 118 may convert the light at a given intensity—which may be directly related to the absorbance and/or reflectance of light in the tissue of the patient 117—into an electrical signal. That is, when more light at a certain wavelength is absorbed or reflected, less light of that wavelength is typically received from the tissue by the detector 118. After converting the received light to an electrical signal, the detector 118 may send the signal to the monitor 102, where physiological characteristics may be calculated based at least in part on the absorption of light in the tissue of the patient 117.

Additionally the sensor 114 may include an encoder 120, which may contain information about the sensor 114, such as what type of sensor it is (e.g., whether the sensor is intended for placement on a forehead or digit) and the wavelengths of light emitted by the emitter 116. This information may allow the monitor 102 to select appropriate algorithms and/or calibration coefficients for calculating the patient's 117 physiological characteristics. The encoder 120 may, for instance, be a memory on which one or more of the following information may be stored for communication to the monitor 102: the type of the sensor 114; the wavelengths of light emitted by the emitter 116; and the proper calibration coefficients and/or algorithms to be used for calculating the patient's 117 physiological characteristics. In one embodiment, the data or signal from the encoder 120 may be decoded by a detector/decoder 121 in the monitor 102.

Signals from the detector 118 and the encoder 120 may be transmitted to the monitor 102. The monitor 102 may include one or more processors 122 coupled to an internal bus 124. Also connected to the bus may be a RAM memory 126, control inputs 110, and the display 104. The processor 122 may also include an audio codec 128 that may be utilized to encode and/or decode a data stream or signal. In one embodiment, the audio codec may be a part of the processor 122. That is, the audio codec 128 may be imbedded or integrated in the processor 122, for example, as part of the same semiconductor die as the processor. Examples of a processor with an integrated audio codec include a Blackfin® series processor by Analog Devices, Inc., such as a Blackfin® ADSP-BF52xC or a BelaSigna® series processor by On Semiconductor Corp. The audio codec 128 may have a frequency response from about 20 Hz-25 kHz with 16-24 bit resolution. In other embodiments, a surround sound DAC may be used in order to obtain more than two output channels.

In one embodiment, the audio codec 128 may include a digital-to-analog converter (DAC) 132 as well as an analog-to-digital converter (ADC) 134. The DAC 132 may operate to generate analog (e.g., light drive) signals that may be used to drive LEDs in the emitter 116 to cause the emitter 116 to emit light into the tissue of a patient 117 so that a patient's 117 physiological characteristics may be measured. The DAC 132 of the audio codec 128 may be directly coupled to the emitter 116 via path 131. In some embodiments, the DAC output may be amplified using, for example a transistor or operational amplifier, in order to provide enough power to drive the emitters at the desired current level (e.g., 50 mA). Some embodiments may use an audio codec 128 with a built-in amplifier provided, for example, to drive headphones but also suitable for driving emitter 116.

Furthermore, the generation of these light drive signals transmitted to the emitter 116 may be controlled by the processor 122 or by the audio codec 128. The light drive signals may control when the emitter 116 is activated and, if multiple light sources are used the multiplexed timing for the different light sources. The digital signal used to drive the DAC 132 may be band-limited to match the input frequency response specification of the DAC (e.g., 20 Hz-25 kHz). This may avoid signal distortion and other problems caused by driving the DAC 132 with an input signal having frequency components outside the DAC's 132 input frequency response. For example, a square wave may have frequency components above and/or below the bandwidth of a DAC 132. Driving a DAC 132 with such a waveform may cause the output analog signal to be distorted in unexpected and/or unpredictable ways. DACs by different manufacturers may have different frequency responses.

The processor 122 may also control the gating-in of signals from detector 118 through a switching circuit 135. These signals from the detector 118 are sampled at the proper time, depending at least in part upon which of multiple light sources is activated, if multiple light sources are used. The received signal from the detector 118 may be passed through an amplifier 136, and a low pass filter 138 to the ADC 134 of the audio codec for amplifying, filtering, and digitizing. The digital data may then be stored in temporary storage, such as a queued serial module (QSM) 140, for later downloading to RAM 126 as the QSM 142 fills up, or for direct use by the processor 122. Additionally, it should be noted that in one embodiment, there may be multiple parallel paths including separate amplifiers and filters for multiple light wavelengths or spectra received, which may be coupled to the ADC 134. In addition, there may be multiple parallel paths including separate amplifiers and filters for the AC and DC portions of a signal.

As with the DAC 132, the signals input to the ADC 134 may be controlled so that they do not contain frequency components outside the manufacturer's specified input frequency bandwidth of the ADC 134. For example, the signals may be limited to an audio bandwidth of 20 Hz-25 kHz. The input signals may be band-limited based on the nature of the LED drive signals provided to the sensor 114 and/or they may be band-limited through preprocessing (such as filtering). As codecs by various manufacturers have different input frequency specifications, the chosen frequency bandwidth will depend on the particular device chosen, as will be understood by one of skill in the art.

The digital data generated by the ADC 134 may be retrieved from the RAM 126 (or from the QSM 140) by the processor 122 and, based at least in part upon the retrieved data (corresponding to the light received by detector 118) processor 122 may calculate the oxygen saturation of a patient 117 using various algorithms. These algorithms may require coefficients, which may be empirically determined. For example, algorithms relating to the distance between an emitter 116 and various detector elements in a detector 118 may be stored in a ROM 142 to be accessed and operated on according to processor 122 instructions. The processor 122 may utilize these algorithms in conjunction with the data retrieved from the RAM 126 to calculate physiological parameters of a patient 117, such as oxygen saturation.

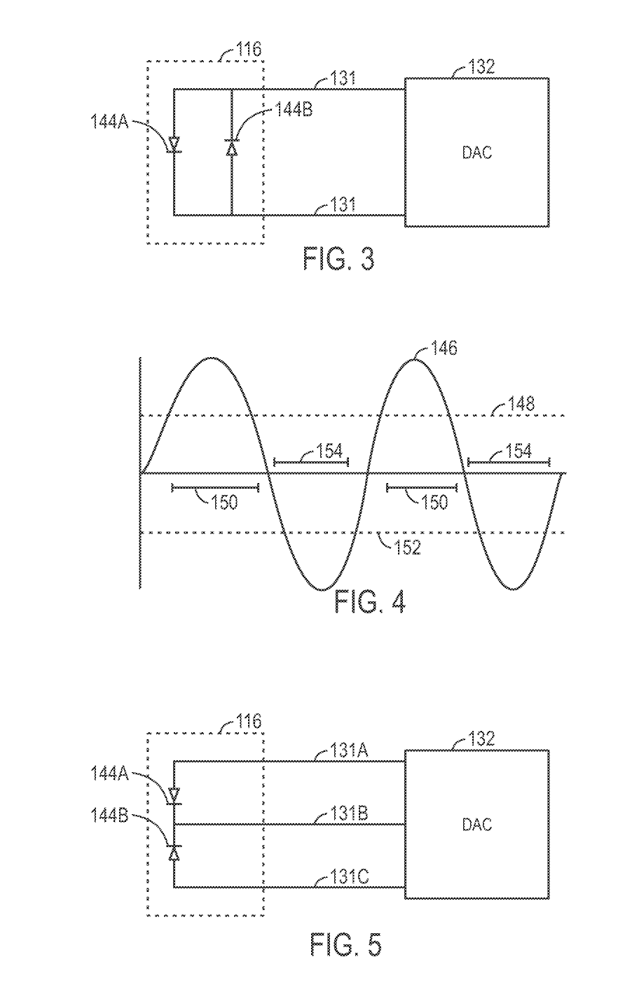

Various types of emitters 116 may be utilized in conjunction with the processor 122. FIG. 3 illustrates an embodiment of an emitter 116 of sensor 114 that may be utilized in conjunction with the monitor 102 of FIG. 2. Emitter 116 is coupled to the DAC 132 of the audio codec 128. The emitter 116 may include a first LED 144A and a second LED 144B. In one embodiment, LED 144A may be a red LED that generates light at a wavelength between about 600 nanometers (nm) and about 700 nm. LED 144B may be an infrared LED that generates light at a wavelength between about 800 nm and about 1000 nm. Use of LEDs that generate light at these wavelengths may be useful for determination of the blood oxygen saturation of the patient 117. Additionally, different LEDs could be used to transmit light at different wavelengths than those discussed above. For example, an LED that transmits light at a wavelength of approximately 1000 nm might be used to determine glucose levels of a patient 117, or an LED that transmits light a wavelength of approximately 550 nm might be used to determine hematocrit levels of a patient 117.

The DAC 132 may generate light drive signals to activate the LEDs 144A and 144B. In one embodiment, the DAC 132 may generate a single signal, such as a sine wave, with a single frequency for transmission to the emitter 116. An illustration of this signal is shown in FIG. 4

FIG. 4 illustrates a light drive signal 146 that is transmitted from the DAC 132 to activate the LEDs 144A and 144B. While the light drive signal 146 is a sine wave, it should be noted that the DAC 132 may generate other types of signals instead of a sine wave. As illustrated, as the light drive signal 146 is positively driven, the signal 146 may cross a positive voltage threshold 148 which causes, for example, LED 144A to activate. LED 144A will remain activated for a time period 150 until the signal 146 crosses the positive voltage threshold 148 again, at which time the LED 144A will cease to generate light. The signal 146 will then cross negative voltage threshold 152 causing LED 144B to activate. LED 144B will remain activated for a time period 154 until the signal 146 crosses the negative voltage threshold 152 again, at which time the LED 144B will cease to generate light. This process may be repeated such that LEDs 144A and 144B are sequentially activated. Thus, the DAC 132 may generate a single light drive signal 146 that may power multiple LEDs 144A and 144B in emitter 116. In another embodiment, the DAC 132 may generate multiple light drive signals concurrently for powering multiple LEDs 144A and 144B in emitter 116.

FIG. 5 illustrates an embodiment of the emitter 116 of sensor 114 that may be used in conjunction with the monitor 102 of FIG. 2 as the DAC 132 generates multiple light drive signals concurrently. As illustrated, emitter 116 is coupled to the DAC 132 of the audio codec 128 and emitter 116 may include a first LED 144A and a second LED 144B. As previously noted, LED 144A may be a red LED that generates light at a wavelength between about 600 nanometers (nm) and about 700 nm and the LED 144B may be an IR LED that generates light at a wavelength between about 800 nm and about 1000 nm. Furthermore, the DAC 132 may generate separate light drive signals to activate each of the LEDs 144A and 144B concurrently.

In one embodiment, the DAC 132 may generate a first signal, such as a sine wave, for transmission to LED 144A of the emitter 116. This first signal may be transmitted along path 131A and may be, for example, a 1000 Hz sine wave. The DAC 132 may also generate a second signal, such as a sine wave, for transmission to LED 144B of the emitter 116. This second signal may be transmitted along path 131B and may be, for example, a 1500 Hz sine wave. Each of LEDs 144A and 144B may also be connected to the DAC 132 via a shared return path such as path 131C.

In one embodiment, the audio codec 128 is capable of generating and transmitting these first and second signals via audio channels, such as a left channel and a right channel. Accordingly, in one embodiment, each of these audio channels may be utilized to drive each of the LEDs 144A and 144B as suggested above. For example, the left channel of the audio codec 128 may be utilized to drive a red light drive signal (e.g., the 1000 Hz sine wave) to a red LED (e.g., LED 144A). Similarly, the right channel of the audio codec 128 may be utilized to drive an IR light drive signal (e.g., the 1500 Hz sine wave) to an IR LED (e.g., LED 144B). In this manner, the DAC 132 may generate separate light drive signals to activate each of the LEDs 144A and 144B concurrently so that multiple physiological parameters of a patient 117 may be measured concurrently. In other embodiments, an audio codec 128 with more than two channels, such as a surround sound codec, may be used in order to drive more than two LEDs.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may not only be applied to measurements of blood oxygen saturation, but these techniques may also be utilized for the measurement and/or analysis of other blood constituents. For example, using the same, different, or additional wavelengths, the present techniques may be utilized for the measurement and/or analysis of carboxyhemoglobin, met-hemoglobin, total hemoglobin, fractional hemoglobin, intravascular dyes, and/or water content. The various embodiments may cover all modifications, equivalents, and alternatives falling within the spirit and scope of the disclosure as defined by the following appended claims.

What is claimed is:

1. A medical monitor comprising:
a processor configured to calculate physiological parameters of a patient based on analog signals indicative of absorption of light in a patient; wherein the processor comprises an audio codec configured such that it receives signals within its input bandwidth.

2. The medical monitor of claim 1, wherein the audio codec comprises a digital-to-analog converter configured to convert digital control signals received from the processor into at least one light drive signal, wherein the digital control signals are within the input bandwidth of the digital-to-analog converter.

3. The medical monitor of claim 2, wherein the at least one light drive signal comprises a sinusoidal signal.

4. The medical monitor of claim 2, wherein the at least one light drive signal comprises first and second sinusoidal signals at different frequencies.

5. The medical monitor of claim 4, wherein the digital-to-analog converter comprises at least two output channels and each of the first and second sinusoidal signals is output by a different output channel.

6. The medical monitor of claim 1, wherein the audio codec comprises a analog-to-digital converter configured to receive analog signals indicative of absorption of light within a patient, wherein the analog signals are within the input bandwidth of the digital-to-analog converter.

7. The medical monitor of claim 6, further comprising pre-processing circuitry for restricting the bandwidth of the analog light absorption signals to be within the input bandwidth of the analog-to-digital converter.

8. A medical device comprising:
a sensor comprising an emitter configured to transmit light through a patient and a detector configured to receive the transmitted light and generate an analog signal indicative of the received transmitted light; and
a processor comprising an audio codec configured to receive signals within its input bandwidth, wherein the processor is configured to:
generate at least one light drive signal;
transmit the at least one light drive signal to the emitter;
receive the analog signal indicative of the received transmitted light from the emitter; and
calculate physiological parameters of a patient based on the analog signal.

9. The medical device of claim 8, wherein the emitter comprises a first light emitting diode configured to transmit light at a first wavelength and a second light emitting diode configured to transmit light at a second wavelength.

10. The medical device of claim 9, wherein the audio codec comprises a digital-to-analog converter configured to receive a digital control signal from the processor and convert the digital control signal into the at least one light drive signal, wherein the digital control signal is within the input bandwidth of the digital-to-analog converter.

11. The medical device of claim 10, wherein the digital-to-analog converter is configured to generate a light drive signal at a single frequency.

12. The medical device of claim 10, wherein the digital-to-analog converter is configured to:
generate a first light drive signal at a first frequency;
transmit the first light drive signal to the first light emitting diode;
generate a second light drive signal at a second frequency different from the first frequency; and
transmit the second light drive signal to the second light emitting diode.

13. The medical device of claim 12, wherein the digital-to-analog converter is configured to generate the first and second light drive signals concurrently.

14. The medical device of claim 8, wherein the audio codec comprises an analog-to-digital converter configured to convert the analog signal indicative of received transmitted light to a digital signal for use by the processor in calculating physiological parameters of the patient.

15. The medical device of claim 14, comprising a switch controlled by the processor and configured to allow the analog signal indicative of received transmitted light to be transmitted to the analog-to-digital converter based on the light drive signal.

16. A method comprising:
generating a digital control signal representative of at least one light drive signal for an emitter, wherein the digital control signal is within the input frequency bandwidth of an audio digital-to-analog converter of an audio codec;
converting the digital control signal to at least one light drive signal using the audio digital-to-analog converter;
transmitting the at least one light drive signal to an emitter to generate light to be transmitted through a patient; and
calculating a physiological parameter of the patient based on the light transmitted through the patient.

17. The method of claim 16, wherein the at least one light drive signal comprises a waveform at a single frequency.

18. The method of claim 16, wherein the at least one light drive signal comprises a first waveform at a first frequency and a second waveform at a second frequency.

19. The method of claim 18, wherein the first waveform is transmitted using a first channel of the audio digital-to-analog converter and the second waveform is transmitted using a second channel of the audio digital-to-analog converter.

20. The method of claim 16, comprising:
receiving an analog signal indicative of light transmitted through a patient, wherein the analog signal is within the input frequency bandwidth of an analog-to-digital converter in the audio codec;
generating a digital data signal in the analog-to-digital converter of the audio codec based on the received analog signal indicative of light transmitted through a patient.
